(19) United States
(12) Patent Application Publication
(10) Pub. No.: US 2023/0415143 A1
(43) Pub. Date: Dec. 28, 2023

(54) SINGLE CELL GEL ELECTROPHORESIS FLOW CYTOMETRY

(71) Applicant: T.C. TRAKYA UNIVERSITESI, Edirne (TR)

(72) Inventor: Utku GUNER, Edirne (TR)

(73) Assignee: T.C. TRAKYA UNIVERSITESI, Edirne (TR)

(21) Appl. No.: 18/253,932

(22) PCT Filed: Nov. 10, 2021
(86) PCT No.: PCT/TR2021/051177

§ 371 Date: May 23, 2023

(30) Foreign Application Priority Data
Nov. 24, 2020 (TR) 2020/18906

(51) Int. Cl. B01L 3/02; G01N 27/447

(52) U.S. Cl. CPC B01L 3/0268; G01N 27/44786; B01L 2200/0647; B01L 2200/0636; B01L 2400/0415; B01L 2400/043; B01L 2200/143; B01L 2300/069

(57) ABSTRACT

A system ensuring the scattering of cells one by one, at a certain speed and direction, on the agarose layer on the lame in SCGE assays is provided. The system includes an electro-magnetic focusing system, a droplet nozzle moving with electro-magnetic guidance, an electric winding creating electromagnetic field for an electro-magnetic charging, an electric motor providing the movement of the lame, an electro-magnetic focusing system, and a control system controlling the electric motor.

### CROSS REFERENCE TO THE RELATED APPLICATIONS

This application is the national phase entry of International Application No. PCT/TR2021/051177, filed on Nov. 10, 2021, which is based upon and claims priority to Turkish Patent Application No. 2020/18906, filed on Nov. 24, 2020, the entire contents of which are incorporated herein by reference.

### TECHNICAL FIELD

The invention relates to the scattering of cells one by one, at a certain speed and direction, on the agarose layer on the lame in SCGE assays.

### BACKGROUND

In the state of the art, the “Single Cell Gel Electrophoresis” (SCGE) technique is a sensitive, fast and inexpensive method used for the detection of DNA breaks. The SCGE technique is also called “Comet Assay” or “Microgel Electrophoretic Technique”.

Although there are many methods used in the said technique, in all methods, the cells used are mixed with low boiling agarose (LMP) (37° C.) with a pipette and transferred onto the agarose layer on the lame. To date, there is no device that automates this process. Cells cannot be transferred individually and regularly onto LMP, cells are randomly scattered on the agarose layer on the lame, and the scattering of cells on the agarose layer on the lame cannot be controlled. How often, how deep and with which pattern the cells will be placed is not known. These problems impose limitations on SCGE analysis.

In SCGE assays, cells are simply scattered onto LMA with pipette. It cannot be predicted how and in what distances the cells will be scattered on the agarose layer on the lame. Shaking the cells does not solve this problem definitively. The lack of a balanced scattering of cells with an appropriate frequency is a major problem in SCGE analysis. Due to this problem, preparations with unsuitable cell scattering and/or some of the preparations cannot be evaluated in SCGE analyses.

### SUMMARY

The present invention relates to a device that allows cells to be scattered on the LMA (agarose) at the appropriate depth and pattern in order to eliminate the above disadvantages and bring new advantages to the related technical field.

This invention provides cells individually onto the LMA by hydrodynamic focusing, how deep the LMA will penetrate is determined based on the electro-magnetic guidance strength. In addition, by means of Electro-magnetic orientation the scattering of cell on the LMA is performed depending on the desired order.

Depending on the user's request, droplet and cell scattering are performed in different patterns and depths.

The present invention places cells at the desired frequency, pattern and appropriate depth on the agarose layer on the lame in SCGE (single cell gel electrophoresis) analyses. In addition, the invention allows performing SCGE analysis with very few cells.

The system allows SCGE analyses to be performed in a fast and reproducible manner and be reliable with less labour.

### BRIEF DESCRIPTION OF THE DRAWINGS

For a better understanding of the invention in question, FIGS. 1-5 and explanations about the drawings are given below.

FIG. 1 Electro-magnetic focusing system

FIG. 2 Droplet nozzle moving with electro-magnetic guidance

FIG. 3 Winding for electromagnetic charging

FIG. 4 Electric motor system providing the movement of the lame

FIG. 5 Basic design of the device

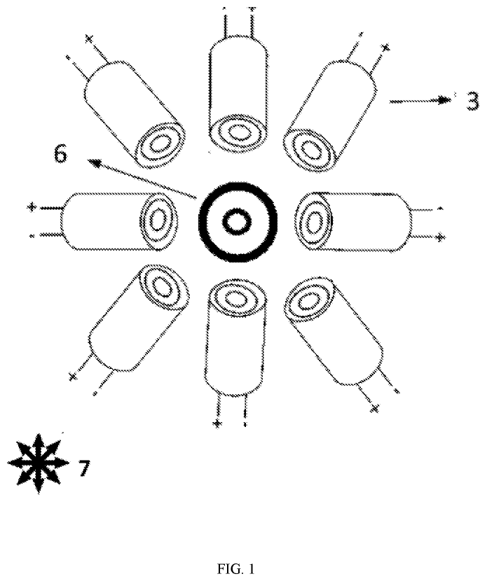

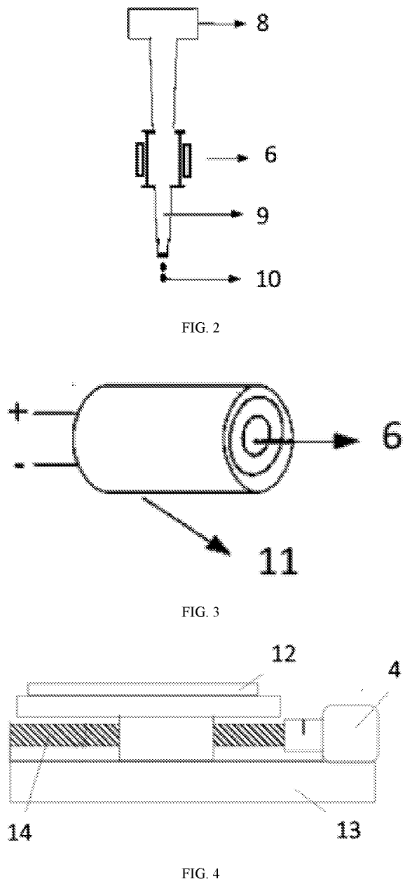

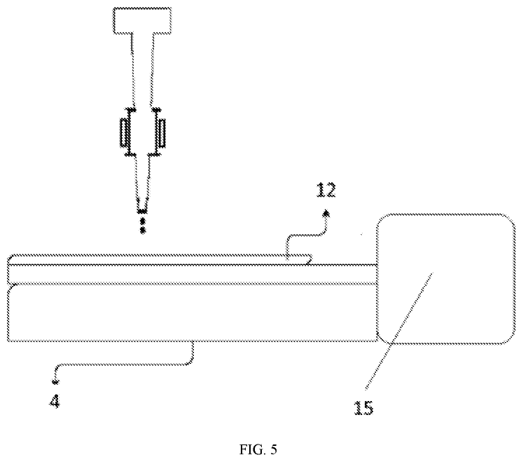

### REFERENCE NUMBERS

For a better understanding of the present invention, the elements and their reference numbers are given below.
1 Electro-magnetic focusing system
2 Droplet nozzles
3 Electric winding
4 Electric motors
5 Basic device
6 Magnetic material
7 Possible directions of movement of the nozzle
8 Flexible connection
9 Nozzle tip
10 Droplet
11 Copper winding
12 LMA Lame
13 Foot
14 Worm screw
15 Control system

### DETAILED DESCRIPTION OF THE EMBODIMENTS

In this detailed explanation, the innovation that is the subject of the invention is only explained for a better understanding of the subject with examples that will not have any limiting effect.

The present invention relates to single cell gel electrophoresis flow cytometry. The invention includes (1) Electro-magnetic focusing system, (1) droplet nozzle moving with electro-magnetic guidance (2), electric winding that creates electromagnetic field for electro-magnetic charging (3), electric motor providing the movement of the lame (4) electro-magnetic focusing system and control system (15) that controls the electric motor.

The present invention guides the cells in the LMA at the appropriate temperature in the electric field with electro-magnetic focusing and sprays them onto the lame that is constantly moving at low speed with an electric motor. The scattering of the cells and their depth in the lame differ depending on the speed of the electric motor, the movement of the moving nozzle due to electro-magnetic focusing, the spray speed in the nozzle, the amount of current sent and current loads of the electric windings that provide electro-magnetic focusing, and in this way, regular and repeatable cell scattering in the desired pattern, frequency and depth are provided.

Based on the detailed explanations above, the present invention is a system that allows cells to scatter on the LMA at the appropriate depth and pattern, including;
An electro-magnetic focusing system (1) that guides the cells in the LMA to be sprayed on the lame which constantly moves at low speed with an electric motor and contains a flexible connection (8) that determines the scattering pattern of the cells coming out of the nozzle, allows the cells to send individual droplets from the nozzle in different directions in x and y coordinates, at the desired depth and at the desired speed, depending on the nozzle movement with electro-magnetic direction,
At least one droplet nozzle (2) with a flexible connection (8) that has magnetic material (6) on it to be used in its movement and enables the droplets to exit and move in x and y axes, and that moves with electro-magnetic guidance,
The electrical winding (3), which provides the electro-magnetic guidance movement of the droplet nozzle (2), and which provides electro-magnetic guidance by determining the different current direction and current amount for electro-magnetic focusing,
At least one electric motor (4), which enables the movement of the lame carrying the LMA, and provides the arrangement of the cell scattering pattern by determining the direction and speed of movement of the lame with the worm screw and electric motor, and
The control system (5) which provides the adjustment of the speed of the electro-magnetic focusing (1) in accordance with the user's desire, and controls the voltage and current directions of the electro-magnetic focusing windings and the electric motor (4) (in terms of movement speed and direction of movement) that moves the lame carrying the LMA.

Other embodiments of the invention are;
A device that allows the cells to be scattered on the LMA at the appropriate (desired) depth and pattern, including the above-mentioned system.
It includes the flexible connection that ensures the arrangement of the cell scattering in the x and y coordinates.
It includes the control system (5) that enables the aforementioned electric motor (4) to be precisely controlled along the length of the lame, in both directions, with very small steps with the worm screw.
Said electric motor (4) is a step motor.
Said scattering of cells and their depth in the lame depend on the speed of the electric motor, the movement of the moving nozzle due to electro-magnetic focusing, the spray speed at the nozzle, the amount of current sent and the current loads of the electric windings that provide electro-magnetic focusing.

What is claimed is:

1. A system ensuring a scattering of cells on an LMA in a desired depth and pattern, comprising;
an electro-magnetic focusing system, wherein the electro-magnetic focusing system guides the cells in the LMA to be sprayed on a lame constantly moving at a low speed with at least one electric motor and containing a flexible connection determining a scattering pattern of the cells coming out of at least one droplet nozzle, and the electro-magnetic focusing system allows the cells to send individual droplets from the at least one droplet nozzle in different directions in x and y coordinates at the desired depth and a desired speed depending on a nozzle movement with an electro-magnetic direction,
the at least one droplet nozzle with the flexible connection, wherein a magnetic material is provided on the at least one droplet nozzles to be used in a movement of the at least one droplet nozzle and enables the individual droplets to exit and move in x and y axes, and the at least one droplet nozzle moves with an electro-magnetic guidance,
an electrical winding, wherein the electrical winding provides the electro-magnetic guidance on the movement of the at least one droplet nozzle by determining different current directions and current amounts for an electro-magnetic focusing,
the at least one electric motor, wherein the at least one electric motor enables a movement of the lame carrying the LMA, and provides an arrangement of the scattering pattern of the cells by determining a direction and a speed of the movement of the lame with a worm screw and the at least one electric motor,
a control system, wherein the control system provides an adjustment of a speed of the electro-magnetic focusing system in accordance with a user's desire, and controls voltage and current directions of electro-magnetic focusing windings and the at least one electric motor in terms of the speed and the direction of the movement of the lame carrying the LMA.

2. The system according to claim 1, comprising the control system enabling a control of the at least one electric motor to move the worm screw in both directions with steps.

3. A device allowing the cells to be scattered on the LMA at the desired depth and pattern, comprising the system according to claim 1.

4. The system according to claim 1, comprising the flexible connection enabling the arrangement of the scattering pattern of the cells in the x and y coordinates.

5. The system according to claim 1, wherein the at least one electric motor is a step motor.

6. The system according to claim 1, wherein the scattering of the cells and the desired depth of the cells in the lame depend on a speed of the at least one electric motor, the movement of the at least one droplet nozzle due to the electro-magnetic focusing, a spray speed at the at least one droplet nozzle, a current amount and current loads of the electrical winding providing the electro-magnetic focusing.
